(19) United States
(12) Patent Application Publication
(10) Pub. No.: US 2018/0117275 A1
(43) Pub. Date: May 3, 2018

(54) Aroma Emitting Pacifier

(71) Applicant: Stephenie Becker, Colorado Springs, CO (US)

(72) Inventor: Stephenie Becker, Colorado Springs, CO (US)

(21) Appl. No.: 15/792,424

(22) Filed: Oct. 24, 2017

Related U.S. Application Data
(60) Provisional application No. 62/413,889, filed on Oct. 27, 2016.

(51) Int. Cl. A61M 21/02; A61J 17/00

(52) U.S. Cl. CPC A61M 21/02; A61M 2021/0016; A61J 17/008; A61J 17/001

(57) ABSTRACT

An aroma emitting pacifier for an infant gives off a predetermined scent by evaporation of volatile essential oils or similar substances. The aroma may be, for example, mint, menthol or eucalyptus, which may help relieve nasal congestion. The aroma emitting pacifier is constructed to prevent evaporation of the essential oil before activation, so that the nipple does not become contaminated with a flavor the infant may find unpalatable, and so that the oil is conserved until it is desired to administer it to the infant.

### CROSS-REFERENCE TO RELATED APPLICATIONS

This is an original U.S. patent application that claims priority to U.S. Provisional Patent Application No. 62/413,889 filed 27 Oct. 2016. The disclosure of that application is specifically incorporated by reference herein for all that it discloses and teaches.

### FIELD

The invention relates to infant and baby products, and to the field of devices that may safely be placed in a baby's mouth. More specifically, the invention relates to an aroma emitting pacifier that can be placed in the mouth of an infant or baby.

### BACKGROUND

The presence of a soft, but relatively impervious object that can be retained in a baby's mouth without the danger of inhalation or swallowing, has long been known to have a calming or pacifying effect on the baby. Such devices are thereby often referred to as pacifiers. Since their introduction, such devices have been improved somewhat in regards to safety, but little else.

As pacifiers are already something that a busy parent is used to ensuring is brought along with, or kept in close proximity to, a baby, introducing an additional functionality within the pacifier can simplify the parent's caretaking requirements. Furthermore, if that additional functionality is difficult to accomplish in any other way, then the enhanced functionality of such a pacifier is particularly valuable.

One such need is for aroma dispensing devices to help babies who are sick or otherwise need to inhale vapors/scents. There are a multitude of vaporizers and humidifiers that attempt to dispense aromas, however, they are often bulky items that do not travel well (if at all) and impact the environment of the entire room rather than dispensing the vapor/aroma just for the baby.

What is needed is a pacifier device that has the additional functionality of dispensing an aroma. Such a device should remain sterile and not introduce any potentially unsanitary conditions. Additionally, it is important that the aroma portion of the pacifier not change the flavor or taste of the nipple portion, as a baby may refuse to utilize a pacifier that has such a modified nipple. Furthermore, the device must be simple to purchase and use, as an already busy parent does not have time to deal with multi-part pacifiers that can come apart, need refilling, or otherwise overly complicate what should remain a relatively simple and effective baby product.

### SUMMARY

One embodiment of the present invention comprises an aroma emitting pacifier having a mouth guard, a pull ring, a top plate, an aroma emitter, and a nipple. The mouth guard is sized large enough to keep the baby from fitting the entire pacifier in his or her mouth (to ensure the pacifier does not become a choking hazard, be swallowed, etc.). A pull ring should attach to the mouth guard so that either baby or parent can grasp the pull ring and maneuver the pacifier. Additionally, a short retention strap or line can be affixed to the pull ring and a nearby object so that if the pacifier is dropped, it does not fall all the way to the ground and become lost or soiled.

Sealing the mouth guard is a top plate which contains the aroma emitter between the top plate and the mouth guard. In an alternate embodiment, the aroma emitter is contained within, and/or is a part of, the top plate and/or the mouth guard. Attached on the bottom side of the mouth guard is a nipple that is designed to be placed within the mouth of the baby.

The aroma emitter is designed to include a repository for an aromatic substance such as medicinal vapor rub(s), fabric soaked in aromatic liquid(s), gel bulbs containing aromatic substance(s), etc. The aroma emitter is initially sealed so that the aromas do not pervade the entire aroma emitting pacifier such that the nipple becomes flavored and the baby rejects it. Instead, the aroma emitter contains the aroma(s) until activated by the parent just prior to giving the pacifier to the baby. Activation can be accomplished in a number of ways. In one embodiment, the parent simply presses down on an aroma emitter activation button which presses downwards on an aroma cavity seal, puncturing the seal, and releasing the aroma(s). In another embodiment, pressing the aroma emitter activation button crushes gel bulb(s) within the aroma cavity and thereby releases aroma(s). Once the aroma(s) are released, they then travel from the aroma cavity, through aroma vents, and out of the aroma emitting pacifier. In yet another embodiment, the vents of the aroma cavity are covered by a seal which is removed when the parent wishes the pacifier to emit the aroma contained in an otherwise unsealed manner within the aroma cavity. Since the baby's nose is in close proximity to the pacifier while the pacifier is being used, relatively small amounts of aroma substance(s) can produce relatively large effects without requiring the air in the entire room to be modified. Once the pacifier is no longer emitting significant amounts aroma(s), it can be disposed of and a new aroma emitting pacifier can be activated and used in its place. In some embodiments, a separate sealed aroma containing cartridge may be provided as a replacement unit. The spent cartridge may be removed and discarded, and a fresh cartridge installed for future use.

### BRIEF DESCRIPTION OF DRAWINGS

The aforementioned and other features and objects of the present invention and the manner of attaining them will become more apparent and the invention itself will be best understood by reference to the following descriptions of a preferred embodiment and other embodiments taken in conjunction with the accompanying drawings, wherein:

FIG. 1 shows a top perspective view of an exemplary embodiment of an aroma emitting pacifier;

FIG. 2 illustrates a rear elevation view of an exemplary embodiment of an aroma emitting pacifier;

FIG. 3 shows a front elevation view of an exemplary embodiment of an aroma emitting pacifier;

FIG. 4 illustrates a left elevation view of an exemplary embodiment of an aroma emitting pacifier;

FIG. 5 illustrates a right elevation view of an exemplary embodiment of an aroma emitting pacifier;

FIG. 6 shows a bottom plan view of an exemplary embodiment of an aroma emitting pacifier;

FIG. 7 shows a top plan view of an exemplary embodiment of an aroma emitting pacifier;

FIG. 8 shows an exploded perspective view of an exemplary embodiment of an aroma emitting pacifier highlighting an aroma emitter and top plate;

FIG. 9 shows an exploded perspective view of an exemplary embodiment of an aroma emitting pacifier highlighting an aroma emitter and top plate;

FIG. 10 illustrates a top perspective view of an exemplary embodiment of an aroma emitting pacifier with the activator button activated and highlighting the aroma emitting vents;

FIG. 11 illustrates a cross-section perspective view showing the interior structures of the aroma emitter including related portions of the aroma cavity, mouth guard, and top plate;

FIG. 12 illustrates a rear elevation view of another exemplary embodiment of an aroma emitting pacifier;

FIG. 13 shows an exploded perspective view of another exemplary embodiment of an aroma emitting pacifier highlighting an aroma emitter and top plate; and

FIG. 14 illustrates a rear elevation view of another exemplary embodiment of an aroma emitting pacifier.

### DETAILED DESCRIPTION

Embodiments of the invention are generally similar to known infant pacifiers, but they comprise novel aroma emitting structures. Some embodiments are single-use or disposable: after activation, and after the aroma or scent dissipates, they may be discarded. Other embodiments comprise a replaceable aroma containing cartridge, which may be exchanged for a new cartridge when one is spent. In all embodiments, the aroma containing structure is sealed or substantially airtight, to prevent premature evaporation of the aromatic substance. In use, the seal is broken or disabled to activate the pacifier and begin emitting the aroma.

In the following discussion, numerous specific details are set forth to provide a thorough understanding of the present disclosure. However, those skilled in the art will appreciate that embodiments may be practiced without such specific details. Furthermore, lists and/or examples are often provided and should be interpreted as exemplary only and in no way limiting embodiments to only those examples.

Exemplary embodiments are described below and in the accompanying Figures. The following detailed description provides a review of the drawing Figures in order to provide a thorough understanding of, and an enabling description for, these embodiments. One having ordinary skill in the art will understand that in some cases well-known structures and functions have not been shown or described in detail to avoid unnecessarily obscuring the description of the embodiments.

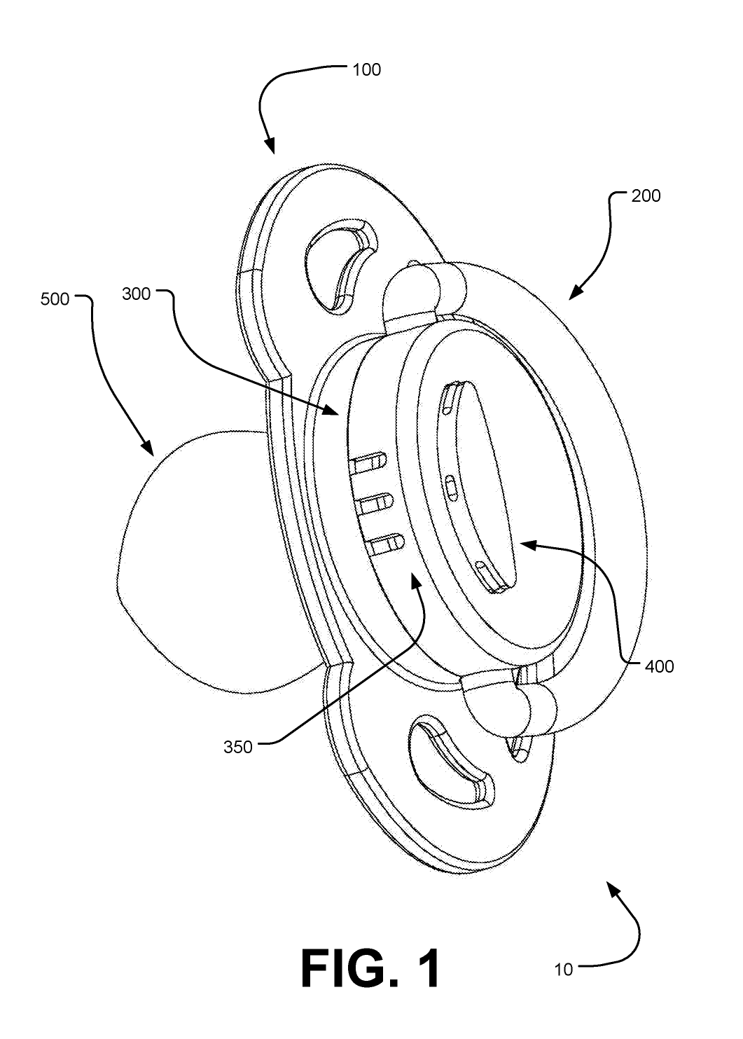

Referring now to the drawings, FIG. 1 shows a top perspective view of an exemplary embodiment of an aroma emitting pacifier 10. As the aroma emitting pacifier 10 is shown from the top and rear, the pull ring 200 and much of the top plate 300 are visible. Some of the mouth guard 100 is hidden behind these features. Similarly, much of the nipple 500 is not visible in FIG. 1. The exposed button activator of the aroma emitter 400 is visible in FIG. 1; however, most of the remaining sub-components of the aroma emitter 400 cannot be seen in this view, see later Drawing Figures for more details.

The mouth guard 100 should be sized large enough to keep the baby from fitting the entire pacifier in his or her mouth. This is desirable because if the pacifier could fit within the baby's mouth, it would become a choking hazard, could be swallowed, etc. There are a number of air ports in the mouth guard 100 illustrated in FIG. 1. These provide attachment points for retention straps/strings, allow the skin that is underneath the mouth guard (when it is in use) to breathe, allow air to pass through the mouth guard, etc. In other embodiments the number, size, shape, and location of these air ports can vary or be non-existent.

An additional attachment point for a strap or retention string can be provided by the pull ring 200. A retention strap/line/string can be affixed to the pull ring and a nearby object so that if the pacifier is dropped, it does not fall all the way to the ground and become lost or soiled. Although not strictly necessary, the pull ring is a useful feature on the aroma emitting pacifier 10 that can be used by either the baby or the parent to grasp and maneuver the pacifier.

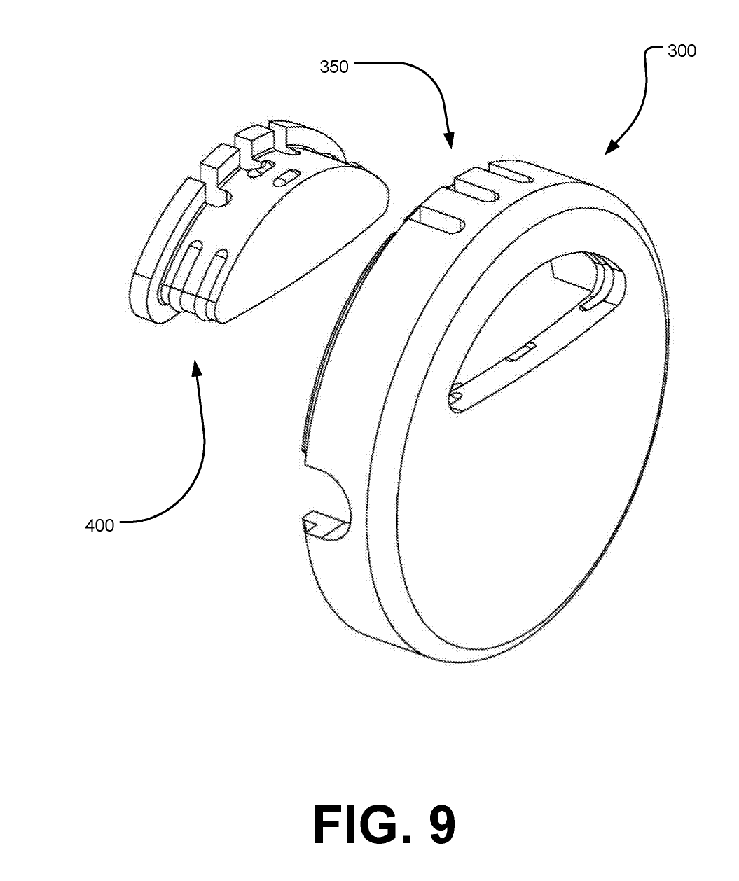

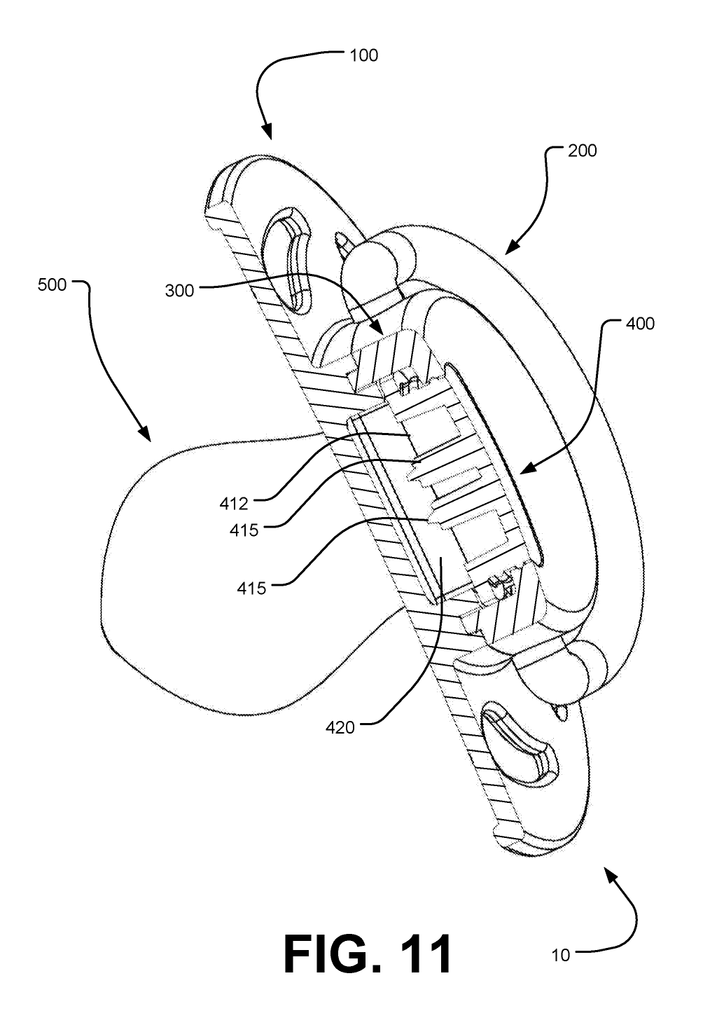

Sealing the mouth guard 100 is a top plate 300 which contains the aroma emitter 400 between the top plate 300 and the mouth guard 100. In an alternate embodiment, the aroma emitter 400 is contained within, and/or is a part of, the top plate 300 and/or the mouth guard 100. In the embodiment shown in FIG. 1, only the aroma emitter activation button portion of the aroma emitter 400 is visible protruding through the top face of the top plate 300. It is in the unactivated position. To activate it, the parent simply presses down on the activation button. A number of small lock ridges can be included on the sides of the activation button. These lock ridges can lock into place within corresponding lock valleys within the top plate (not shown in FIG. 1, see FIGS. 9 and 11) so that, once pressed, the activation button is retained within the top plate and cannot be pressed again. In an alternate embodiment, a spring or some similar type of device can be included to pop the activation button back up once pressed, thereby providing the parent with another opportunity to press the activation button. In such an embodiment, lock ridges/lock valleys may be reduced in size and/or eliminated completely.

The aroma emitter 400 is designed to include a repository for an aromatic substance such as medicinal vapor rub(s), fabric soaked in aromatic liquid(s), gel bulbs containing aromatic substance(s), etc. Other materials that can be doused with aromatic substances include wood chips, cotton balls, strings, sponges, and any other suitable material that can initially retain and then release aromatic substances over time.

A variety of different aromatic substances may be used in an aroma emitting pacifier according to an embodiment of the invention. These substances should be non-toxic and have at least a moderate degree of volatility so that they evaporate when the seal of the aroma emitter is broken to activate the pacifier. Embodiments comprising one or more of the following substances are specifically contemplated: lavender, orange, lemon, eucalyptus, peppermint, rosemary, almond oil, coconut oil, camphor, cedar, menthol, tea tree oil, vanilla, chamomile, and cannabis. These may be pure essential oils, tinctures prepared from oils and alcohols, or other similar forms where the aromatic substance evaporates when a substantially-airtight seal is broken or removed.

The aroma emitter 400 is initially sealed so that the aromas do not pervade the entire aroma emitting pacifier 10 such that the nipple 500 becomes flavored and the baby rejects it. Instead, the aroma emitter 400 contains the aroma(s) until activated by the parent just prior to giving the pacifier to the baby. Activation can be accomplished in a number of ways. In one embodiment, the parent simple presses down on the aroma emitter activation button (as described above), which presses downwards on an aroma cavity seal, puncturing the seal, and releasing the aroma(s). In another embodiment, pressing the aroma emitter activation button crushes gel bulb(s) within the aroma cavity and thereby releases aroma(s). In other embodiments, other release (activation) mechanisms are contemplated. A seal between the aroma substance and the activation button can comprise a foil, plastic, paper, rubber, silicon, gel or similar type of material (or combination) that is glued onto, melted in place, ultrasonically welded to, or otherwise affixed over and/or around the aromatic substance to contain it until the activation button is pressed to release the aroma. The rate of release (e.g., by evaporation) of the aromatic substance may be controlled by providing a labyrinthine channel through which the aromatic substance must travel to exit the cavity. The length and cross-section of this channel may be adjusted to account for the volatility of the aromatic substance, the quantity of substance in the chamber or cavity, and the length of time over which the aroma emitting pacifier is desired to operate.

Once the aroma(s) are released, they then travel from the aroma cavity, through aroma vents (see those in the top plate 300 illustrated in FIG. 1), and out of the aroma emitting pacifier 10. Since the baby's nose is in close proximity to the pacifier 10 while the pacifier is being used, relatively small amounts of aroma substance(s) can produce relatively large effects without requiring the air in the entire room to be filled with aroma. Once the pacifier is no longer emitting significant amounts of aroma(s), the old aroma emitting pacifier can be disposed of and a new aroma emitting pacifier can be activated and used in its place. In embodiments with a replaceable aroma cartridge, the spent cartridge can be removed and replaced with a new one.

Attached on the bottom side of the mouth guard is a nipple that is designed to be placed within the mouth of the baby. While the baby is sucking on and/or gumming the nipple, the aroma emitting pacifier is constantly releasing aroma(s) in close proximity to the baby's nose and mouth. Although designed to be inhaled by the nose, when the nose is plugged and/or the baby is breathing through his or her mouth, the aromas emitted by the aroma emitting pacifier can be inhaled through the mouth as well.

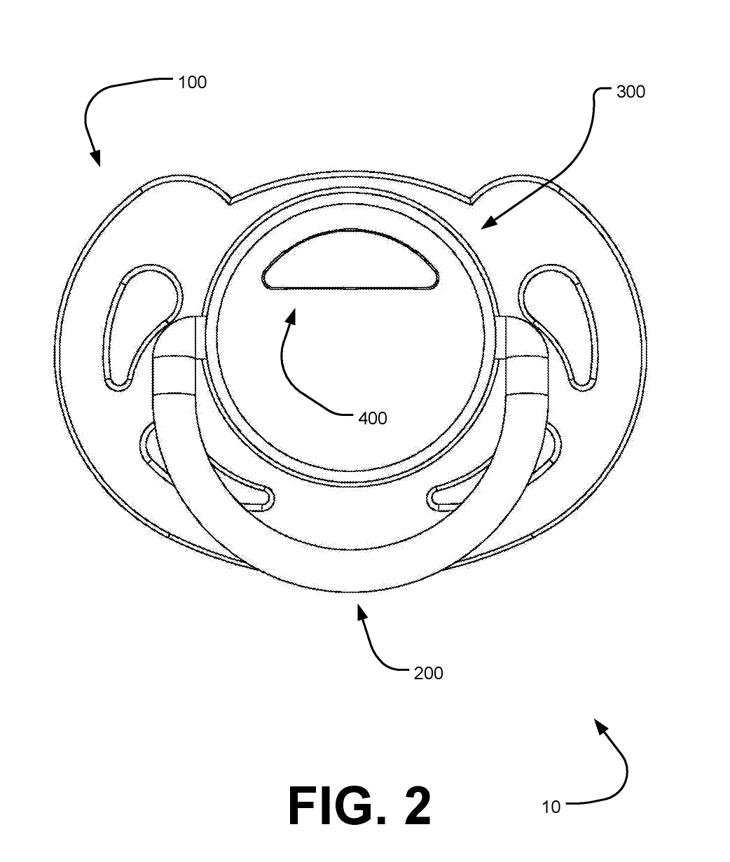

FIG. 2 illustrates a rear elevation view of an exemplary embodiment of an aroma emitting pacifier 10. The pull ring 200 is visible as is the outer surface of the top plate 300. The activator button portion of the aroma emitter 400 can also be seen. The larger size of the mouth guard 100 relative to these other components is obvious in this view. The nipple 500 is not visible as it is hidden behind the other components from this angle.

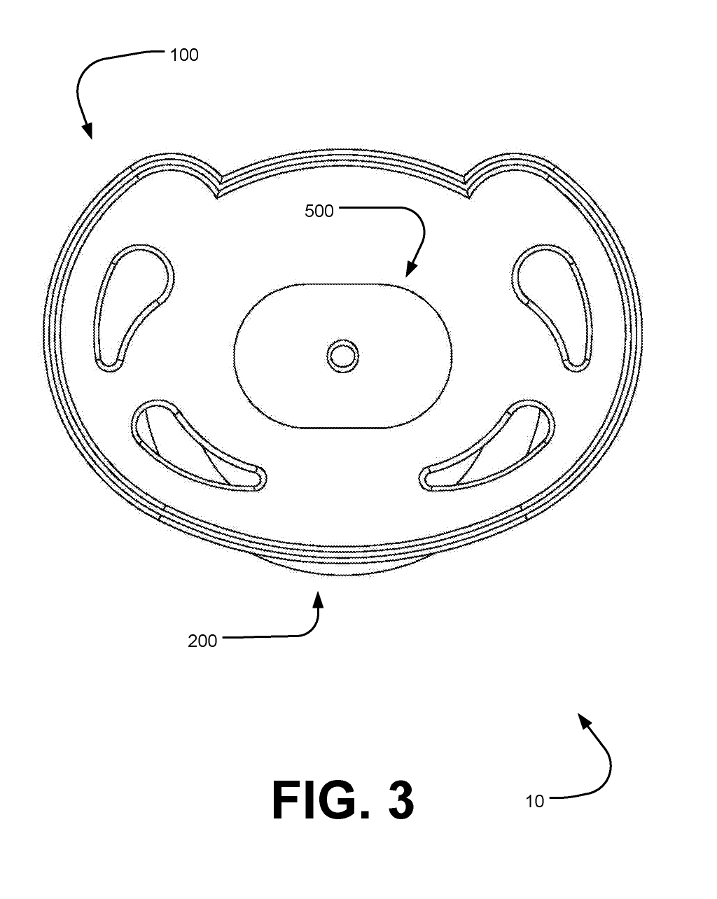

FIG. 3 shows a front elevation view of an exemplary embodiment of an aroma emitting pacifier 10. As this view is simply a hundred and eighty degree horizontal rotation of the pacifier from FIG. 2, the top plate 300, aroma emitter 400, and most of the pull ring 200 are not visible here. Instead, the front face of the mouth guard 100 and nipple 500 are shown.

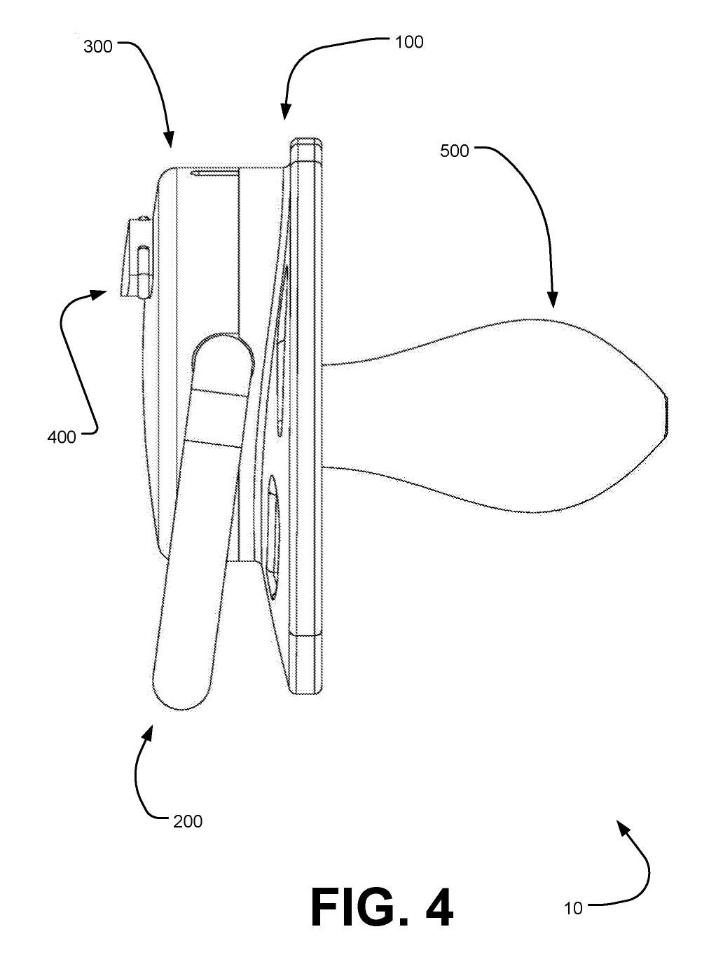

FIG. 4 illustrates a left elevation view of an exemplary embodiment of an aroma emitting pacifier 10. In this view, the shape and protruding nature of the nipple 500 are apparent. Also, note how the aroma emitter activator button 400 protrudes above the surface of the top plate 300; the button must be in its not-yet-activated state in this view. Although only one of the aroma vents in the top plate 300 is visible in this view, it is easy to visualize how close to the baby's nose the aroma vents would be when the nipple is fully engaged within the baby's mouth and the pull ring 200 is hanging downwards towards the baby's chin. In the embodiment illustrated in FIG. 4, the aroma vents are shown on the top surface of the top plate, directly under the baby's nose when the pacifier is in the baby's mouth. In other embodiments, the aroma vents can be located in other locations on the top plate, on the mouth guard, or on the pull ring. In yet another embodiment, one or more combinations of these various locations for the aroma vents can be utilized. The number, size and shape of the aroma vents can also be varied in other embodiments.

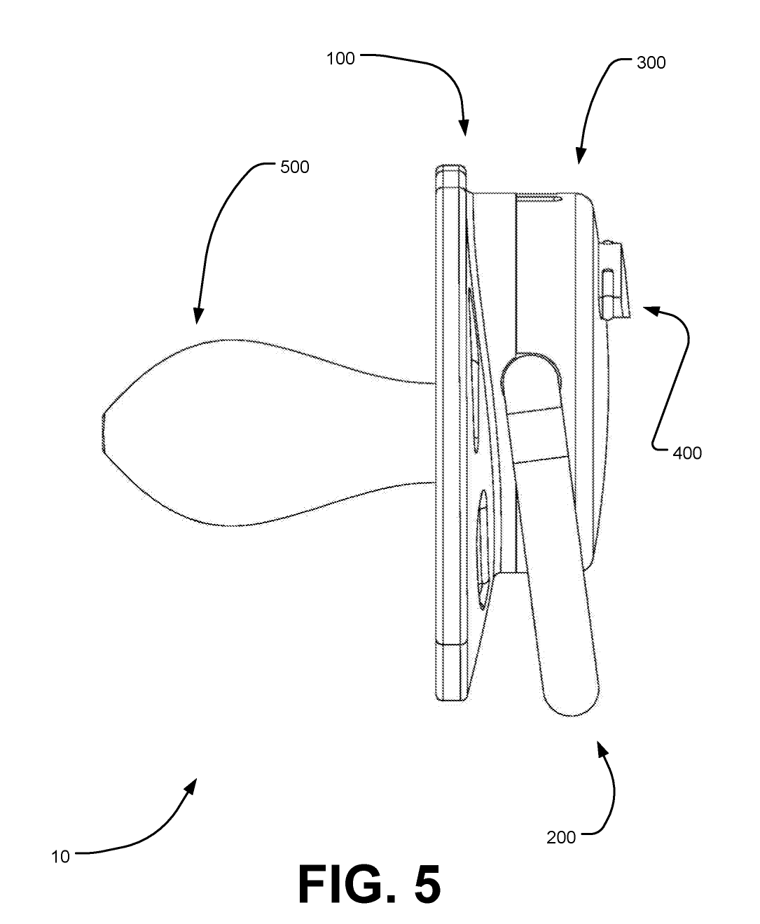

FIG. 5 illustrates a right elevation view of an exemplary embodiment of an aroma emitting pacifier 10. In this particular embodiment, the right elevation view shown in FIG. 5 is basically a mirror image of the left view from FIG. 4. In other embodiments, the aroma emitting pacifier 10 may not be mirrored left and right.

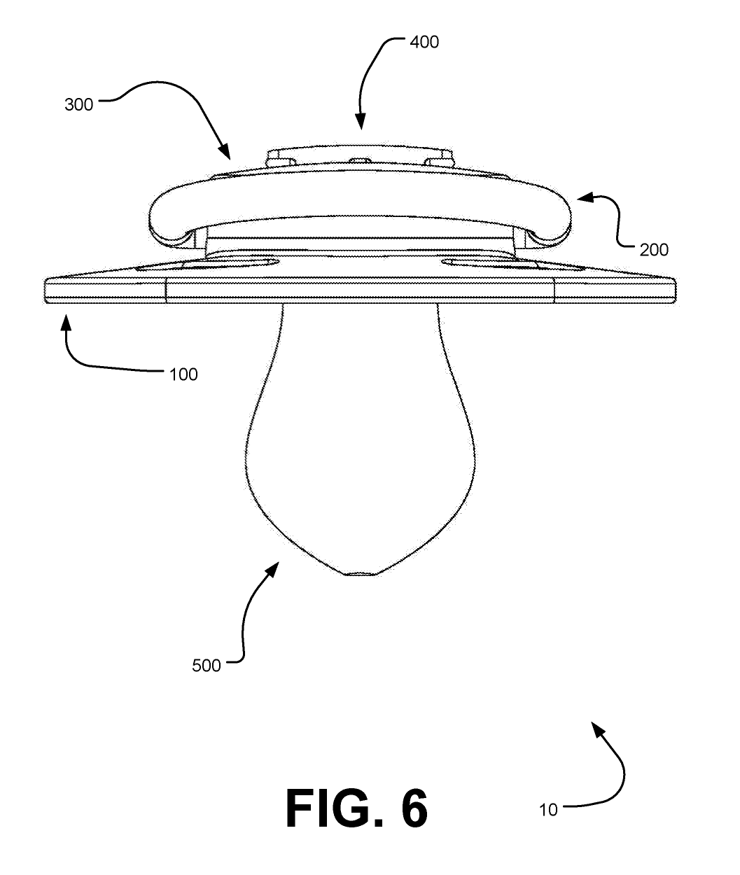

FIG. 6 shows a bottom plan view of an exemplary embodiment of an aroma emitting pacifier 10. In this view, the pull ring 200 is mostly visible since it hangs downwards to the bottom when not being actively grabbed. In another embodiment, the pull ring 200 is employs a stiffer attachment method and so it does not automatically swing downwards when no longer in use.

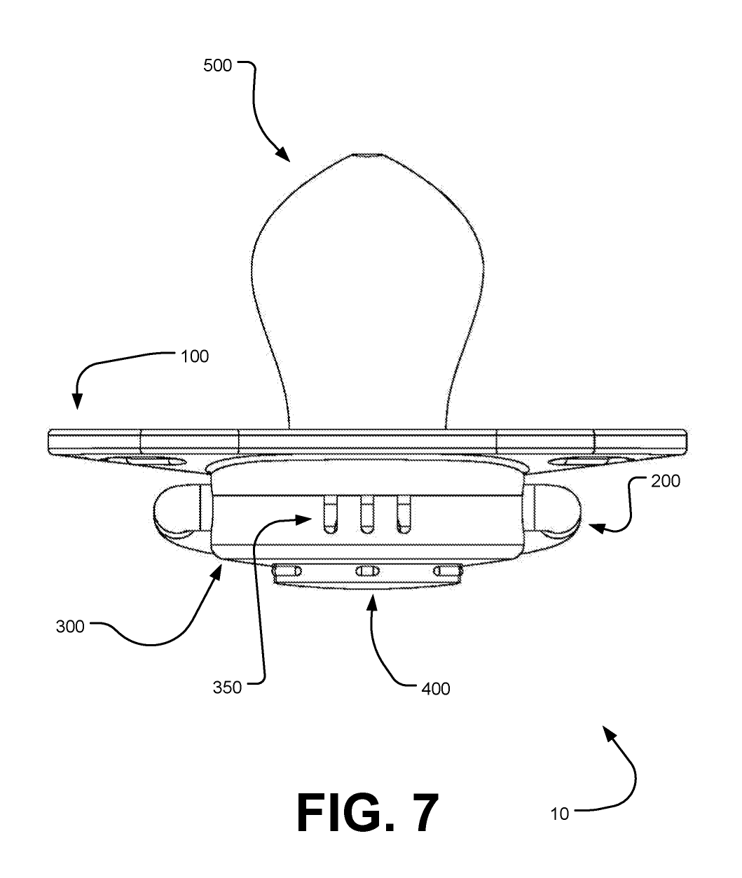

FIG. 7 shows a top plan view of an exemplary embodiment of an aroma emitting pacifier 10. In this view, the plurality of aroma vents 350 in the upper surface of the top plate 300 are visible. In other embodiments, more or fewer aroma vents can be utilized.

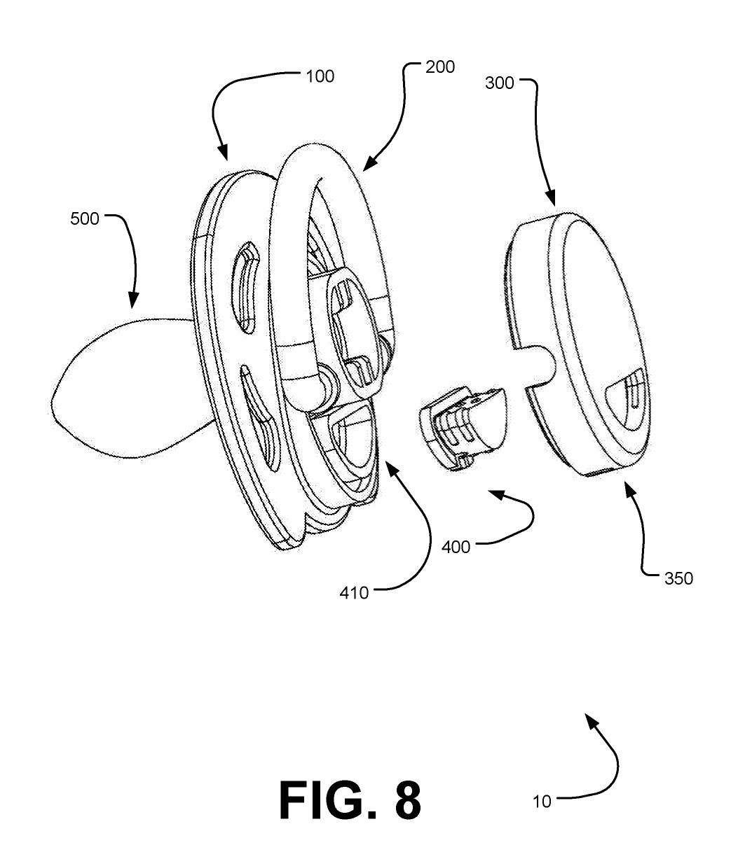

FIG. 8 shows an exploded perspective view of an exemplary embodiment of an aroma emitting pacifier 10 highlighting an aroma emitter 400 and top plate 300. Note how the activator button extends through the top plate when the components are pressed back together. Also, note the aroma cavity 410 illustrated in the mouth guard 100. It comprises the half-circle shaped cavity over which the activator button would fit when pressed together. In other embodiments, the aroma cavity 410 is built into the button itself rather than employing a structure within the mouth guard. In yet another embodiment, the aroma cavity can be attached to or built into the top plate. In yet another embodiment, the top plate can be integral with the mouth guard and the aroma cavity 410 attached thereto or be formed therein. In any case, it allows for placement therein of aroma substances which are released upon activation of the activator button and released to the baby for inhalation. In all embodiments, the aromatic substance is substantially sealed within the aroma cavity until the pacifier is activated by puncturing a seal of the cavity, by peeling away a film adhered over the cavity, by crushing or disrupting a capsule containing the aromatic substance, or by another similar action. Preferably, the aroma cavity cannot be resealed after it is opened once. This offers a tamper-evident function so that parents can be assured that the pacifier and its contents have not been adulterated or modified since manufacture.

FIG. 9 shows an exploded perspective view of an exemplary embodiment of an aroma emitting pacifier 10 highlighting an aroma emitter 400 and top plate 300. This is a close up view of the components highlighted in FIG. 8 and shows the lock ridges and corresponding lock valleys discussed earlier in more detail. Also, the aroma vents in the top surface and corresponding aroma vents in the aroma emitter can be seen as well. For more of the internal structure and other components comprising the aroma emitter, see FIG. 11.

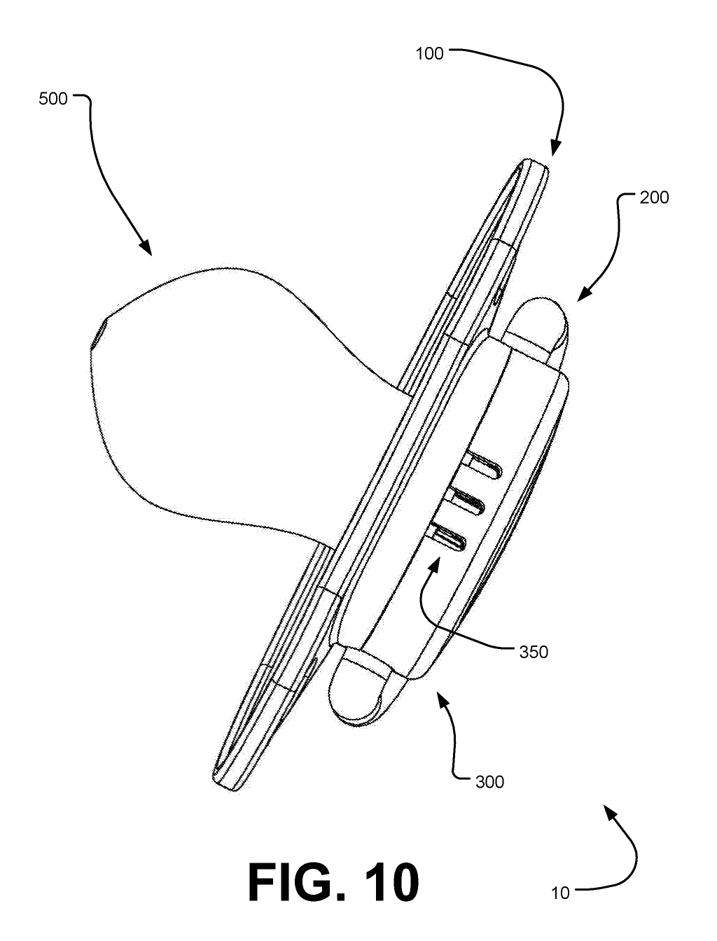

FIG. 10 illustrates a top perspective view of an exemplary embodiment of an aroma emitting pacifier 10 with the activator button activated and highlighting the aroma emitting vents. Since the aroma emitter has had the activator button pressed, it is contained within the top plate 300 and so is not visible in this view. In other embodiments that utilize a multi-press activator button, the button can pop back out of the top plate after activation and would be visible in this view for those embodiments.

FIG. 11 illustrates a cross-section perspective view showing the interior structures of the aroma emitter 400 including related portions of the aroma cavity 410, mouth guard 100, and top plate 300. Since the activator button has been pressed in this view, a plurality of activator protrusions 415 can be seen extending into the aroma cavity 410, having broken through the aroma cavity seal 412, and thereby releasing the aromas from the aromatic substance 420 within the aroma cavity. The aromas can then travel through the aroma vents in the aroma emitter 400 (see FIG. 9), through the aroma vents in the top plate 350 (see FIG. 9), and out to the baby for inhalation.

Note also the structure and resulting function of the lock ridges and lock valleys visible in FIG. 11. It should be apparent that when the activator button is in the not-yet-activated position, a first set of lock ridges on the activator button are in place within the lock valleys of the top plate 400. Then, when the parent presses down on the activator button, the first set of lock ridges pop out of the lock valleys, allowing the activator button to move downwards. After the activator protrusions break through the aroma cavity seal, the second set of lock ridges pop into place within the lock valleys on the top plate, retaining the activator button in its activated position. As discussed above, a multi-press activator button can be utilized in other embodiments. Such a multi-press button could pop back up into the not-yet-activated state and be pressed again, if desired.

The mechanical arrangements by which the aromatic substance is released from the sealed chamber are not critical, and many different approaches will be evident to one of ordinary skill. For example, instead of a separate button that punctures the sealed chamber, as shown in FIG. 11, the activator protrusions may be coupled to the pull ring 200, so that the pacifier is activated by moving the pull ring fully to one extreme and forcing the activator protrusions (“lances”) to break the substantially-airtight aroma cavity seal.

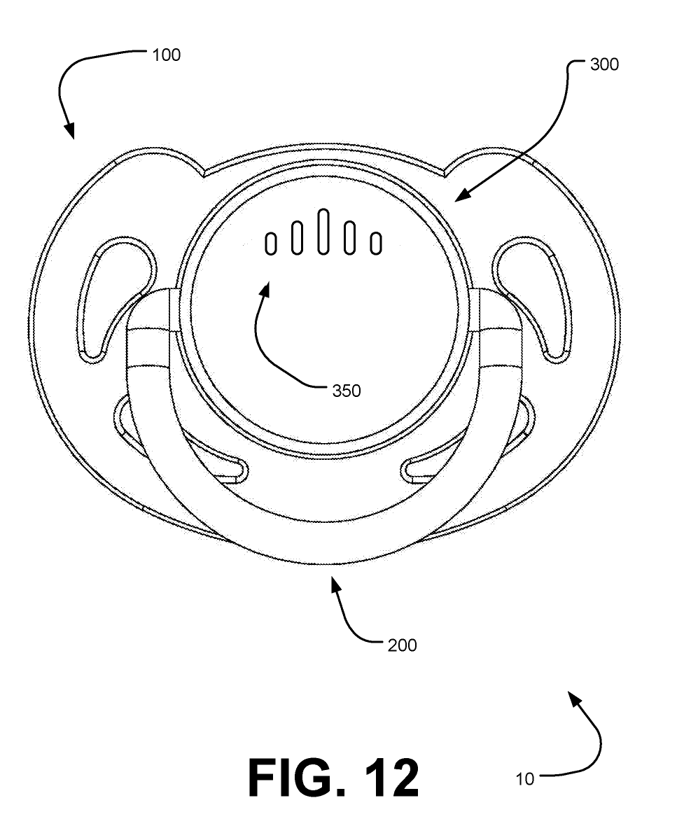

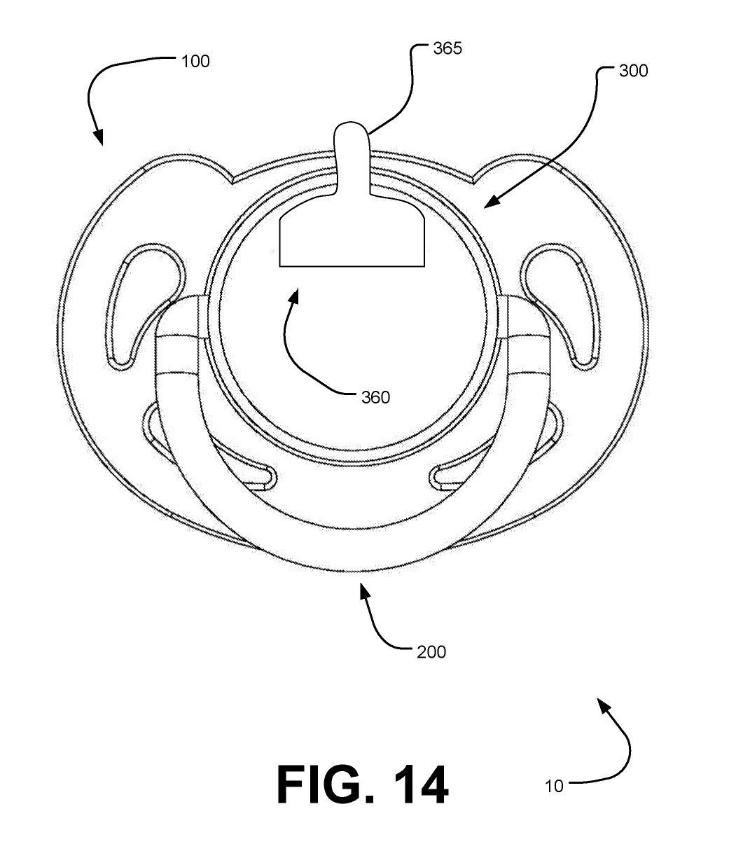

FIG. 12 illustrates a rear elevation view of another exemplary embodiment of an aroma emitting pacifier 10. In this embodiment, a multi-press activator button is not used. Instead, the aroma cavity has rear facing aroma vents 350 that release the aroma from the aroma cavity 410 when an aroma vent seal 360 (not shown in FIG. 12, see FIG. 14) is removed from the aroma vents 350. In this alternate embodiment, no button is necessary since the aroma vent seal 360 is simply peeled away by the user to activate the aroma emitting functions of the pacifier 10. This arrangement may also be provided with a tamper-evident feature: the seal 360 may comprise a fragile, colored layer which is disrupted when the seal is removed. Thus, an attempt to re-seal a “new” pacifier (e.g., after tampering) would be immediately apparent.

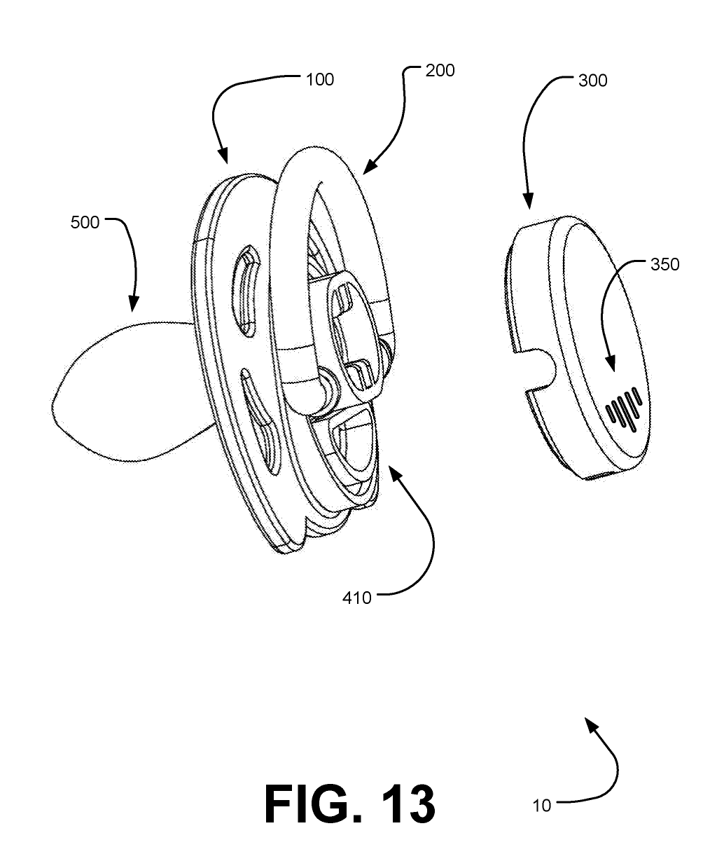

FIG. 13 shows an exploded perspective view of another exemplary embodiment of an aroma emitting pacifier 10 highlighting an aroma emitter and top plate 300. Note that in this alternate embodiment, aroma vents 350 can be on the rear surface of the top plate 300, instead of, or in addition to, the top surface of the top plate as shown in earlier FIGs. In this embodiment, the aroma cavity 410 is illustrated as a half-circle walled protrusion on the rear face of the mouth guard. In other embodiments, the aroma cavity 410 can be built into the top plate 300 wherein the walled protrusion extends downwards therefrom and seals against the mouth guard when attached in place. Regardless of the particular configuration, the aroma cavity can contain an aromatic substance directly (instead of utilizing a gel bulb or other replaceable sub-container for the aromatic substance).

FIG. 14 illustrates a rear elevation view of another exemplary embodiment of an aroma emitting pacifier 10. In this illustration, the aroma vent seal 360 is shown in place, sealing the aroma vents. To make it easy for the parent to remove the aroma vent seal 360, the seal can incorporate a pull tab 365 that is simple for the parent to grasp and peel the seal 360 from the top plate 300.

While particular embodiments of the invention have been described and disclosed in the present application, it should be understood that any number of permutations, modifications, or embodiments may be made without departing from the spirit and scope of this invention. Accordingly, it is not the intention of this application to limit this invention in any way except as by the appended claims.

Particular terminology used when describing certain features or aspects of the invention should not be taken to imply that the terminology is being redefined herein to be restricted to any specific characteristics, features, or aspects of the invention with which that terminology is associated, unless specifically noted. In general, the terms used in the following claims should not be construed to limit the invention to the specific embodiments disclosed in the specification, unless the above “Detailed Description” section explicitly defines such terms. Accordingly, the actual scope of the invention encompasses not only the disclosed embodiments, but also all equivalent ways of practicing or implementing the invention.

The above detailed description of the embodiments of the invention is not intended to be exhaustive or to limit the invention to the precise embodiment or form disclosed herein or to the particular field of usage mentioned in this disclosure. While specific embodiments of, and examples for, the invention are described above for illustrative purposes, various equivalent modifications are possible within the scope of the invention, as those skilled in the relevant art will recognize. Also, the teachings of the invention provided herein can be applied to other systems, not necessarily the system described above. The elements and acts of the various embodiments described above can be combined to provide further embodiments.

In light of the above “Detailed Description,” the inventor may make changes to the invention. While the detailed description outlines possible embodiments of the invention and discloses the best mode contemplated, no matter how detailed the above appears in text, the invention may be practiced in a myriad of ways. Thus, implementation details may vary considerably while still being encompassed by the spirit of the invention as disclosed by the inventor. As discussed herein, specific terminology used when describing certain features or aspects of the invention should not be taken to imply that the terminology is being redefined herein to be restricted to any specific characteristics, features, or aspects of the invention with which that terminology is associated, unless so specified.

While certain aspects of the invention are presented below in certain claim forms, the inventor contemplates the various aspects of the invention in any number of claim forms. Accordingly, the inventor reserves the right to add additional claims after filing the application to pursue such additional claim forms for other aspects of the invention.

The above specification, examples and data provide a description of the structure and use of exemplary implementations of the described articles of manufacture and methods. It is important to note that many implementations can be made without departing from the spirit and scope of the invention.

I claim:

1. An aroma emitting pacifier, comprising:
a nipple of suitable construction to be placed in an infant's mouth, said nipple coupled to a mouth guard sized to prevent the aroma emitting pacifier from entirely entering the infant's mouth;
an aromatic substance sealed in a substantially airtight chamber within the aroma emitting pacifier; and
means for breaking a seal of the substantially airtight chamber so that the aromatic substance can exit the substantially airtight chamber.

2. The aroma emitting pacifier of claim 1 wherein the seal of the substantially airtight chamber is formed by at least one of:
a hermetic seal;
an ultrasonic weld; and
a labyrinth seal.

3. The aroma emitting pacifier of claim 1 wherein the seal is tamper-evident so that it cannot be undetectably resealed after being broken once.

4. The aroma emitting pacifier of claim 1 wherein the means for breaking the seal is at least one of:
a lance to puncture the substantially airtight chamber;
a removable adhesive film covering an opening in the substantially airtight chamber; and
an impact member to disrupt a shell of the substantially airtight chamber.

5. The aroma emitting pacifier of claim 1 wherein the aromatic substance comprises at least one of menthol oil, eucalyptus oil, camphor, vanilla, mint, rosemary oil and lavender oil.

6. The aroma emitting pacifier of claim 1 wherein the aromatic substance comprises at least one of orange oil, lemon oil, almond oil, coconut oil, tea tree oil, chamomile essence and cannabis oil.

7. The aroma emitting pacifier of claim 1 wherein a vent through which the aromatic substance exits the substantially airtight container is near a nose of the infant when the nipple is in the infant's mouth.

8. The aroma emitting pacifier of claim 1 wherein a top plate is attached to the mouth guard, the top plate comprises a substantially airtight chamber, and the airtight chamber is separate and discrete from the nipple and mouth guard, said substantially airtight chamber being replaceable with a fresh substantially airtight chamber containing an aromatic substance.

9. The aroma emitting pacifier of claim 1 wherein the substantially airtight chamber is integrated with the nipple and mouth guard so that the aroma emitting pacifier substantially ceases to emit the aroma after most of the aromatic substance exits the substantially airtight chamber.

10. An aroma emitting pacifier having an inactive state and an active state, comprising:
a nipple of suitable construction to be placed in an infant's mouth, said nipple coupled to a mouth guard sized to prevent the aroma emitting pacifier from fully entering the infant's mouth;
a volatile aromatic substance contained within the aroma emitting pacifier, said volatile aromatic substance treated so that it cannot evaporate in the inactive state; and
means for impairing a treatment of the volatile aromatic substance so that it can evaporate in the active state.

11. The aroma emitting pacifier of claim 10, wherein the volatile aromatic substance is treated by sealing it within a capsule, and wherein the means for impairing the treatment is at least one of:
a mechanism for rupturing the capsule; and
a mechanism for puncturing the capsule.

12. The aroma emitting pacifier of claim 10, wherein the volatile aromatic substance is treated by sealing it within a capsule, the aroma emitting pacifier further comprising:
a covered chamber sized to accept the capsule.

13. The aroma emitting pacifier of claim 11 wherein the capsule is a first capsule, the aroma emitting pacifier further comprising:
a second sealed capsule containing the volatile aromatic substance, said second sealed capsule suitable to replace the first capsule after the volatile aromatic substance in the first capsule has evaporated.

14. An aroma emitting pacifier, comprising:
a nipple of suitable construction to be placed in an infant's mouth, said nipple coupled to a mouth guard sized to prevent the aroma emitting pacifier from entirely entering the infant's mouth; and
an aromatic substance stored in a ventilated chamber within the aroma emitting pacifier, said ventilated chamber sealed by a removeable, airtight film covering an opening of the ventilated chamber.

15. The aroma emitting pacifier of claim 14 wherein the opening of the ventilated chamber is near the infant's nose when the nipple is placed in the infant's mouth.

16. The aroma emitting pacifier of claim 14 wherein the removeable, airtight film is tamper-evident.

17. The aroma emitting pacifier of claim 14 wherein the aromatic substance is one of eucalyptus oil, mint oil and vanilla.

18. The aroma emitting pacifier of claim 14 further comprising a pull ring attached to the mouth guard and configured as a grasp handle for holding onto the aroma emitting pacifier.

19. The aroma emitting pacifier of claim 14 further comprising a top plate that is attached to the mouth guard, and wherein the ventilated chamber is located within the top plate.

20. The aroma emitting pacifier of claim 19, wherein the top plate further comprises a plurality of aroma vents that provide a passage from the ventilated chamber and through the top plate so that the aromatic substance can travel therethrough to reach the nose of the infant.
